(19) United States
(12) Patent Application Publication
(10) Pub. No.: US 2016/0310405 A1
(43) Pub. Date: Oct. 27, 2016

(54) Insect Repellent and Suntan Composition and System

(71) Applicants: ANA CARRO, PONCE, PR (US); DEVERAL BODDEN, Ponce, PR (US)

(72) Inventors: ANA CARRO, PONCE, PR (US); DEVERAL BODDEN, Ponce, PR (US)

(21) Appl. No.: 15/000,430

(22) Filed: Jan. 19, 2016

Related U.S. Application Data
(60) Provisional application No. 62/106,896, filed on Jan. 23, 2015.

(51) Int. Cl. A61K 8/97; A61Q 13/00; A61Q 19/00; A61K 8/92; A61K 8/368; A61K 8/81; A01N 65/28; A01N 65/36; A01N 65/08; A61K 8/67; A61K 8/34; A61K 8/27; A61Q 17/04; A01N 65/22

(52) U.S. Cl. CPC A61K 8/97; A61Q 17/04; A61Q 13/00; A61Q 19/007; A61K 8/922; A61K 8/368; A01N 65/22; A01N 65/28; A01N 65/36; A01N 65/08; A61K 8/675; A61K 8/673; A61K 8/676; A61K 8/342; A61K 8/345; A61K 8/27; A61K 8/8147; A61K 2800/49; A61K 2800/524; A61K 2800/5922

(57) ABSTRACT

Disclosed is a topical insect repellent composition and method utilizing a mixture of vitamins and extracts of lemon, coffee and/or peppermint as active insect deterrents. Exemplary embodiments disclosed herein may be utilized to repel insects for hours after application to the skin of both humans and animals. The exemplary embodiments may provide this repellant without the harmful effects of other chemicals and repellants.

### CROSS-REFERENCE TO RELATED APPLICATIONS

This application claims priority to co-pending U.S. Provisional patent application Ser. No. 62/106,896 titled “Insect Repellent and Suntan Composition and System”, filed on Jan. 23, 2014 the disclosure of which is herein incorporated by reference in its entirety.

### Patents Cited

The following documents and references are incorporated by reference in their entirety, Bodden et al (U.S. Pat. No. 8,691,299), Mills (U.S. Pat. Pub. No. 2007/0092544) and Enscore et al (U.S. Pat. No. 4,559,222).

### FIELD OF THE INVENTION

The present invention generally relates to the field of insect repellents, and in particular to an insect repellant composition, system and method which in addition to provide insect repellant functions, provides additional benefits in sun protection and skin care.

### DESCRIPTION OF THE RELATED ART

Most insect repellents use chemicals that may be harmful to humans and animals. Even when they are ‘naturally derived’, the delivery method for them tend to rely heavily in intrusive mixtures or atomizers. These chemicals and their delivery method sometimes have harmful effects upon humans, animals, and other. What is needed is repellent that utilizes natural ingredients that may be ingested by a human or animals as part of their everyday activities (preferably with additional vitamins and nutrients), such that its use (and benefits) becomes part of everyday life, and occurs without harmful effects.

### SUMMARY OF THE INVENTION

This section is for the purpose of summarizing some aspects of the present invention and to briefly introduce some preferred embodiments. Simplifications or omissions may be made to avoid obscuring the purpose of the section. Such simplifications or omissions are not intended to limit the scope of the present invention.

The present invention is for an insect repellent composition and method utilizing a mixture of vitamins and extracts of lemon, coffee and/or peppermint as active insect deterrent. In one aspect, the invention is about a sunscreen, humectant and insect repellent stable emulsion composition, comprising: a sunscreen agent group present in an amount effective to provide an SPF of from about 0.01 wt % to about 0.10 wt %; an insect repelling agent group present in an amount from about 0.3 wt % to about 0.8 wt %; a skin catalyst agent group present in an amount from about 0.5 wt % to about 1.0 wt %; a humectant agent group present in an amount from about 0.3 wt % to about 0.8 wt %; an emulsifying agent for forming a stable emulsion present in an amount from about 4 wt % to about 8 wt %; a balance of an aqueous solvent; and wherein said composition has the capability of repelling insects.

In another aspect, said sunscreen agent group is comprised of at least one of; zinc oxide, octyl methoxycinnamate, octyl salicylate, and/or oxybenzone; said insect repelling agent group is comprised of at least one of; lemon, eucalyptus, coffee and/or peppermint extracts; said skin catalyst agent group is comprised of at least one of; Vitamin B1, Vitamin B2, Vitamin B6, Vitamin B12, Pantothethic acid, Biotin, Vitamin C and/or Folic Acid; said skin humectant agent group is comprised of at least one of; Jojoba, Argan, Aloe Vera and/or Mint extracts; and said emulsifying agent is comprised of Cetyl alcohol, glycerin and Eumulgin. In yet another aspect, at least one fragrance in an amount from about 0.3 wt % to about 0.8 wt %; at least one emollient in an amount of about 0.04 wt %; and at least one preservative in the amount of 0.02 wt %. In another aspect, said fragrance is comprised of at least one of a group comprised of; Vanilla, almond, coconut, lemon, baby lotion (similar to that of JOHNSON AND JOHNSON BABY LOTION, other tropical fruits and/or their combination; said emollient is at least one of a group comprised of; Eumulgin; and said preservative is at least one of a group comprised of; Sodium Benzoate.

In one aspect, the invention is about a method for preparing a sunscreen humectant and insect repellent composition comprising the steps of: providing a sunscreen agent group present in an amount effective to provide an SPF of from about 0.01 wt % to about 0.10 wt %; providing an insect repelling agent group present in an amount from about 0.3 wt % to about 0.8 wt %; providing a skin catalyst agent group present in an amount from about 0.5 wt % to about 1.0 wt %; providing a humectant agent group present in an amount from about 0.3 wt % to about 0.8 wt %; providing an emulsifying agent for forming a stable emulsion present in an amount from about 4 wt % to about 8 wt %; combining the above ingredients with an aqueous solution to form a stable emulsion.

Other features and advantages of the present invention will become apparent upon examining the following detailed description of an embodiment thereof, taken in conjunction with the attached drawings.

### BRIEF DESCRIPTION OF THE DRAWINGS

FIG. 1 shows an illustration of a transdermal patch in the Prior Art.

The above-described and other features will be appreciated and understood by those skilled in the art from the following detailed description, drawings, and appended claims.

### DETAILED DESCRIPTION OF THE PREFERRED EMBODIMENT

This section is for the purpose of summarizing some aspects of the present invention and to briefly introduce some preferred embodiments. Simplifications or omissions may be made to avoid obscuring the purpose of the section. Such simplifications or omissions are not intended to limit the scope of the present invention.

To provide an overall understanding of the invention, certain illustrative embodiments and examples will now be described. However, it will be understood by one of ordinary skill in the art that the same or equivalent functions and sequences may be accomplished by different embodiments that are also intended to be encompassed within the spirit and scope of the disclosure. The compositions, apparatuses, systems and/or methods described herein may be adapted and modified as is appropriate for the application being addressed and that those described herein may be employed in other suitable applications, and that such other additions and modifications will not depart from the scope hereof.

Simplifications or omissions may be made to avoid obscuring the purpose of the section. Such simplifications or omissions are not intended to limit the scope of the present invention. All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

As used in the specification and claims, the singular forms “a”, “an” and “the” include plural references unless the context clearly dictates otherwise. For example, the term “a transaction” may include a plurality of transaction unless the context clearly dictates otherwise. As used in the specification and claims, singular names or types referenced include variations within the family of said name unless the context clearly dictates otherwise.

Certain terminology is used in the following description for convenience only and is not limiting. The words “lower,” “upper,” “bottom,” “top,” “front,” “back,” “left,” “right” and “sides” designate directions in the drawings to which reference is made, but are not limiting with respect to the orientation in which the modules or any assembly of them may be used.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

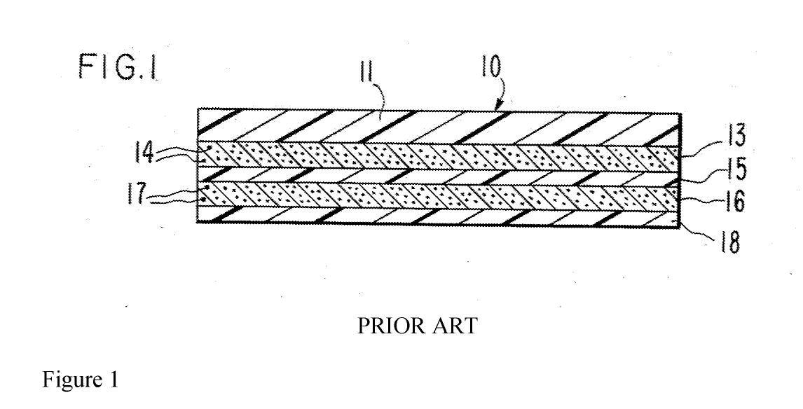

Most of us are very familiar with transdermal patches designed to infuse medication into the blood stream. FIG. 1 illustrates such a patch structure, according to the Prior Art disclosure in Enscore et al (U.S. Pat. No. 4,559,222). In their various embodiments, such patches pass a certain element, component or combination of them through the skin layer, into the bloodstream. In this fashion, medication can be administered to a user externally.

The invention described here enhances the above by applying to the body skin one or more skin catalyst agents that have been found to have insect repellent qualities, and by combining them with a number of catalysts that facilitate body actions such as circulation, achieving a formulation that will encourage the interaction of the natural occurring insect repelling agents to the skin surface, from where the natural evaporation or sublimation of the agent in the user's skin provides for its action as a repellent.

The repellent agent attaches itself to the skin or dermis of the person receiving the topical application, having in addition the benefits of reducing the effect of dangerous ultraviolet sun radiation, as well as natural humectant agents that provide skin hydration and other beneficial effects. That is, the user applies an agent, element, or combination of agents that then attach themselves to the skin and interact with the perspiration, sweat or other fluid transfer to the skin. This fluid secretion by the skin is common in mammals as a cooling mechanism. Even in some animals with no sweat gland, it is common for fluids to be secreted through their mouth.

In short, the disclosed system allows for a non-toxic repellent to be combined with the sweat. A common reaction to sweating, is to use the palms of our hands to distribute it along the skin (as well as to say, “phew, its hot”, although the latter has no contribution to the repellent action), this spreading of the sweat, does has the effect of coating any area of the body that has not had any repellent coverage, increasing its effectiveness.

The skin hydrating and/or humectant agent elements may be comprised of one or a combination of the following elements; Jojoba, Argan, Mint, Aloe and/or similar human skin friendly oils that allow for their benefits to the skin when included in a topical cream or lotion.

Jojoba with the botanical name Simmondsia chinensis, and also known as goat nut, deer nut, pignut, wild hazel, quinine nut, coffeeberry, and gray box bush, is native to Southwestern North America. Simmondsia chinensis is the sole species of the family Simmondsiaceae, placed in the order Caryophyllales. Argan oil is a plant oil produced from the kernels of the argan tree (Argania spinosa L.). Aloe is a genus containing over 500 species of flowering succulent plants. The most widely known species is Aloe Vera, or “true aloe”, so called because, though probably extinct in the wild, it is cultivated as the standard source of so-called “aloe vera” for assorted pharmaceutical purposes.

In addition to many of the other agents having benefits as sun protection, in another embodiment adding one or more direct sunscreen or sun-blocking agent elements (such as Zinc oxide, titanium dioxide and/or other mixtures thereof) provides for additional SPF protection (typically in the 30 SPF range, although any range from 5 to 75 or more SPF may be achieved). Some of the other sun screening agents include include octyl methoxycinnamate, oxybenzone, amino benzoic acid, Cinoxate, DEA-methoxycinnamate, Digalloyl, Dioxybenzene, Padimate O, Ethyl dihydroxypropyl p-aminobenzoate, octyl salicylate, glyceryl aminobenzoate, Homosalate, Urocanic acid, isopropylbenzyl salicylate, menthyl anthranilate, octocrylene, Sulisbenzone and its sodium salt and triethanolamine salicylate.

The insect repelling agent portion of the mixture basis lies in a suitable skin activation agent (any of a number of substances that are capable of activating the blood vessels near the human dermis) that remains safe from long term effects to the user and their skin. Such activation agent(s) must be completely safe to the human (or animal) subjected to the topical application of said elements. Some of the repellent agents have crossing capabilities as humectants and repellents. However, the preferred activation agents may be comprised of one or a combination of the following elements; lemon, eucalyptus, coffee, peppermint and/or others.

When we refer to the lemon, we are not referring to the small evergreen tree (Citrus x limon, often given as C. limon) native to Asia, but to said tree's oval yellow fruit. The fruit is used for culinary and non-culinary purposes throughout the world, primarily for its juice, though the pulp and rind (zest) are also used in cooking and baking. Lemon juice is about 5% to 6% (approximately 0.3 M) citric acid, which gives lemons a sour taste, and a pH of 2 to 3. This makes lemon juice an inexpensive, readily available acid for use in educational science experiments. Many lemon-flavored drinks and candies are available, including lemonade and sherbet lemons. In one embodiment, the extract of said lemon juice is used, in concentrations of at least 10:1.

Eucalyptus is a diverse genus of flowering trees and shrubs (including a distinct group with a multiple-stem mallee growth habit) in the myrtle family, Myrtaceae. Members of the genus dominate the tree flora of Australia. Eucalyptus is one of three similar genera that are commonly referred to as “eucalypts”, the others being Corymbia and Angophora.

Coffee is a brewed drink prepared from roasted seeds, called coffee beans, of the coffee plant. These beans are the seeds of coffee cherries that grow on said tree. Coffee has a stimulant effect, and can have a stimulating effect on humans due to its caffeine content. However, applicant's experiments have shown positive results with both Caffeinated and Decaffeinated coffee extracts. In one embodiment, the extract of said coffee is used in concentrations of at least 10:1.

Peppermint (Menthe x pipe Rita, also known as M. balsa mea Wild) is a hybrid mint, formed from a cross between the water mint (Menthe aquatic) and spearmint (Menthe spiced). It is found wild occasionally with its parent species. Peppermint has a high menthol content, and its oil also contains menthone and menthyl esters, particularly menthyl acetate. Dried peppermint typically has 0.3-0.4% of volatile oil containing menthol (29-48%), menthone (20-31%), menthyl acetate (3-10%), menthofuran (1-7%) and many trace constituents including limonene, pulegone, eucalyptol, and pinene. In one embodiment, the peppermint leaf extract is used in concentrations of at least 10:1, and as high as 100:1.

Some of the other insect repelling agents that have been found to be natural insect repellents include; Achillea alpine, alpha-tyerpinene, basil, callicarpa Americana or Beautyberry, Camphor, castor oil, Catnip, cedar oil, celery extract, cinnamon, clove, fennel, garlic, geranium, lavender, lemongrass, marigold, rosemary and thyme. Of course, these various materials work best when reduced to some oil or concentrate, also in a 10:1 (or higher) concentration.

In order to maximize said the effectiveness of the other selected agents, in one embodiment, a skin catalyst agent group, comprised of a combination of B vitamins (also known as B-complex vitamins) and Vitamin C is added to the repelling agent described above. B vitamins are a group of water-soluble vitamins that play important roles in cell metabolism. B vitamins were once thought to be a single vitamin, then referred to as vitamin B. Later research showed that they are chemically distinct vitamins, although they often coexist in the same foods. Supplements containing all eight are referred to as a vitamin B complex, while Individual B vitamin supplements are referred to by the specific name of each vitamin (e.g., B₁, B2, B₆, C, etc.).

In one embodiment, the repellent agent is combined with a skin catalyst agent (comprised of one or more B complex vitamin groups), said catalyst groups formed from a combination of individual B vitamins from a group comprised from said vitamin B complex. They have the effect of accelerating the user's metabolism, facilitating the exuding of fluids across the dermis (usually as part of sweating). The sweating has the added advantage of distributing any cream and/or oils across the skin. In one embodiment, this is done by adding quantities of vitamins B₁ (Thiamine), B₂ (Riboflavin), B₆ (Pyridoxine, pyridoxal, or pyridoxamine, or pyridoxine hydrochloride)) and B₁₂ (Cobalamins or Cyanocobalamin).

In one embodiment, the ratios of repellent agent to catalyst group vitamins (B₁, B₂, B₆ and/or B₁₂) is in the 1000:1 to 20000:1, with a 7000:1 ratio found to be effective. In effect, in one embodiment, the selected B complex catalyst group ‘encourages’ your body to sweat, something fairly common when encountering mosquitoes, which tend to exist in high humidity environments. Various combinations are possible, with the key being an up to 33% variation in a roughly 1750:1 repellent agent to totality of catalyst group. Various catalyst groups' combinations are possible, with an up to 33% variation on equal parts of B₁, B₂, B₆, and B₁₂.

As for their dilution in fluids, the various examples seen below provide an idea of mixing ratios, with the main effect in the amount of fluid being the duration of protection. In general approximately 350 mg of repellent agent diluted in 240 ml, are sufficient for 2 to 6 hours of protection.

In addition to the above skin catalyst agents, other vitamins and elements such as those comprising, the remaining B-complex members, including B₃ (niacin or niacinamide), B₅ (Pantothenic acidor Calcium d-Pantothenate), B₇ (Biotin) and B₉ (Folic Acid), Vitamin C or Ascorbic Acid, as well as stability agents such as Potassium Sorbate in various ratios may be added.

In one embodiment, the invention is made into a cream by the addition of an aqueous solvent (e.g. purified water or similar) and one or more emulsifiers (e.g. Cetyl alcohol) at certain temperatures. A further improvement would include the addition of skin humectant agents (in order to keep the skin hydrated and/or moist) and a sun blocker, making the mix into one more suitable for external application directly to the skin.

One or more emulsifying agents may be added for forming a stable emulsion. Said emulsifiers may belong to a group similar to that to which the group comprised by Cetyl alcohol, Glycerin, Eumulgin and others.

A typical process for this includes the premixing of an emulsifier alcohol (such as Cetyl) at approximately 82 deg. C. into water, ensuring the alcohol is fully diluted, then adding the other ingredients. The mixture is then mixed for about 15 minutes and then mixed at 1,000 rpm until the mixture becomes a cream, at which point it may be bottled into appropriate containers.

Appropriate fragrances may be added, including those from the group comprised by; Vanilla, almond, coconut, lemon, baby lotion (similar to that of JOHNSON AND JOHNSON BABY LOTION, other tropical fruits and/or their combination. Other ingredients may include Sodium Benzoate and Carbomer.

Compositions according to the following formulae were prepared;

### Example 1

Composition One: A premix of Humectant agents;

272 grams of jojoba extract;

272 grams of argan extract;

60 grams of Aloe Vera Extract;

136 grams of peppermint extract;

### Example 2

Composition Two: A premix of Skin Catalyst Agents;

| | | |
|---|---|---|
| Bioting | 25 | mcg |
| Folic Acid | 25 | mcg |
| Niacin | 25 | mg |
| Pantothenic Acid | 25 | mcg |
| Vitamin B₁ | 25 | mcg |
| Vitamin B₁₂ | 25 | mcg |
| Vitamin B₆ | 25 | mcg |
| Vitamin B₂ | 25 | mcg |
| Vitamin C | 250 | mg |

### Example 3

Composition Three: A mix for a sunscreen, insect repellent and humectant stable emulsion composition. As an example such a mix could be made with the following suggested ratios;

130.6 Kgs. of Water

4.54 Kgs. of Cetyl alcohol

2.72 Kgs. of Glycerin;

1.68 Kgs. of Eumulgin;

1.13 Kgs of Catalyst agents

862 grams of Repellant agents

740 grams of Humectant agents

720 grams of 50/50 fragrances;

60 grams of sodium benzoate;

60 grams of Zinc Oxide extract; and

30 grams of Carbomer;

The above may be varied, so that by weight percentage (wt %) the sunscreen agent group present is a range of about 0.01 wt % to about 0.10 wt %; the insect repelling agent group present is in a range of about 0.3 wt % to about 0.8 wt %; the skin catalyst agent group is in a range of about 0.5 wt % to about 1.0 wt %; the humectant agent group present in is in a range of about 0.3 wt % to about 0.8 wt %; the emulsifying agent is in a range of about 4 wt % to about 8 wt %; and the rest is a balance of aqueous solutions.

In one embodiment, mixing the above may be accomplished through the following method steps; adding the Cetyl alcohol to the water, aiding the dissolving of the alcohol in the water by having it at 180 F, adding the remaining ingredients (making the total 40 gals) and mixing for 15 minutes in a mixing tank at 1,000 rpm until forming a cream. Cooling said cream mixture to ambient temperature and filling 4 oz. containers, then optionally measuring the degrees of BRIX, Ph, STD Viscosity, keeping Good Manufacturing Practices and FDA records.

### CONCLUSION

In concluding the detailed description, it should be noted that it would be obvious to those skilled in the art that many variations and modifications can be made to the preferred embodiment without substantially departing from the principles of the present invention. Also, such variations and modifications are intended to be included herein within the scope of the present invention as set forth in the appended claims. Further, in the claims hereafter, the structures, materials, acts and equivalents of all means or step-plus function elements are intended to include any structure, materials or acts for performing their cited functions.

It should be emphasized that the above-described embodiments of the present invention, particularly any “preferred embodiments” are merely possible examples of the implementations, merely set forth for a clear understanding of the principles of the invention. Any variations and modifications may be made to the above-described embodiments of the invention without departing substantially from the spirit of the principles of the invention. All such modifications and variations are intended to be included herein within the scope of the disclosure and present invention and protected by the following claims.

The present invention has been described in sufficient detail with a certain degree of particularity. The utilities thereof are appreciated by those skilled in the art. It is understood to those skilled in the art that the present disclosure of embodiments has been made by way of examples only and that numerous changes in the arrangement and combination of parts may be resorted without departing from the spirit and scope of the invention as claimed. Accordingly, the scope of the present invention is defined by the appended claims rather than the forgoing description of embodiments.

We claim:

1. a sunscreen, humectant and insect repellent stable emulsion composition, comprising:
a sunscreen agent group present in an amount effective to provide an SPF of from about 0.01 wt % to about 0.10 wt %;
an insect repelling agent group present in an amount from about 0.3 wt % to about 0.8 wt %;
a skin catalyst agent group present in an amount from about 0.5 wt % to about 1.0 wt %;
a humectant agent group present in an amount from about 0.3 wt % to about 0.8 wt %;
an emulsifying agent for forming a stable emulsion present in an amount from about 4 wt % to about 8 wt %;
a balance of an aqueous solvent; and
wherein said composition has the capability of repelling insects.

2. The composition of claim 1, wherein:
said sunscreen agent group is comprised of at least one of; zinc oxide, octyl methoxycinnamate, octyl salicylate, and/or oxybenzone;
said insect repelling agent group is comprised of at least one of; lemon, eucalyptus, coffee and/or peppermint extracts;
said skin catalyst agent group is comprised of at least one of; Vitamin B1, Vitamin B2, Vitamin B6, Vitamin B12, Pantothethic acid, Biotin, Vitamin C and/or Folic Acid;
said skin humectant agent group is comprised of at least one of; Jojoba, Argan, Aloe Vera and/or Mint extracts; and
said emulsifying agent is comprised of Cetyl alcohol, glycerin and Eumulgin.

3. The composition of claim 2, further comprising:
at least one fragrance in an amount from about 0.3 wt % to about 0.8 wt %;
at least one emollient in an amount of about 0.04 wt %; and
at least one preservative in the amount of 0.02 wt %.

4. The composition of claim 3 wherein;
said fragrance is comprised of at least one of a group comprised of; Vanilla, almond, coconut, lemon, baby lotion (similar to that of JOHNSON AND JOHNSON BABY LOTION, other tropical fruits and/or their combination;
said emollient is at least one of a group comprised of; Eumulgin; and
said preservative is at least one of a group comprised of; Sodium Benzoate.

5. A method for preparing a sunscreen humectant and insect repellent composition comprising the steps of:
providing a sunscreen agent group present in an amount effective to provide an SPF of from about 0.01 wt % to about 0.10 wt %;
providing an insect repelling agent group present in an amount from about 0.3 wt % to about 0.8 wt %;
providing a skin catalyst agent group present in an amount from about 0.5 wt % to about 1.0 wt %;
providing a humectant agent group present in an amount from about 0.3 wt % to about 0.8 wt %;
providing an emulsifying agent for forming a stable emulsion present in an amount from about 4 wt % to about 8 wt %;
combining the above ingredients with an aqueous solution to form a stable emulsion.

6. The method of claim 5, wherein:
said sunscreen agent group is comprised of at least one of; zinc oxide, octyl methoxycinnamate, octyl salicylate, and/or oxybenzone;
said insect repelling agent group is comprised of at least one of; lemon, eucalyptus, coffee and/or peppermint extracts;
said skin catalyst agent group is comprised of at least one of; Vitamin B1, Vitamin B2, Vitamin B6, Vitamin B12, Pantothethic acid, Biotin, Vitamin C and/or Folic Acid;
said skin humectant agent group is comprised of at least one of; Jojoba, Argan, Aloe Vera and/or Mint extracts; and
said emulsifying agent is comprised of Cetyl alcohol, glycerin and Eumulgin.

7. The method of claim 6, further comprising:
at least one fragrance in an amount from about 0.3 wt % to about 0.8 wt %;
at least one emollient in an amount of about 0.04 wt %; and
at least one preservative in the amount of 0.02 wt %.

8. The method of claim 7 wherein;
said fragrance is comprised of at least one of a group comprised of; Vanilla, almond, coconut, lemon, baby lotion (similar to that of JOHNSON AND JOHNSON BABY LOTION, other tropical fruits and/or their combination;
said emollient is at least one of a group comprised of; Eumulgin; and
said preservative is at least one of a group comprised of; Sodium Benzoate.
